(19) United States
(12) Patent Application Publication
(10) Pub. No.: US 2007/0031450 A1
(43) Pub. Date: Feb. 8, 2007

(54) Stabilizers for veterinary vaccines

(75) Inventors: Mahesh Kumar, Fort Dodge, IA (US); Jeff H. Rodenberg, Fort Dodge, IA (US); Jason M. Hansen, Omaha, NE (US); Andrew D. Chu, Fort Dodge, IA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/496,201

(22) Filed: Jul. 31, 2006

Related U.S. Application Data
(60) Provisional application No. 60/705,508, filed on Aug. 4, 2005.

(51) Int. Cl. C12Q 1/68; A61K 39/12

(52) U.S. Cl. 424/204.1; 435/6

(57) ABSTRACT

A formulation for a animal vaccines is provided. The vaccine formulation contains a stabilizer component and a viral immunogen. The stabilizer component includes a substantially TSE/BSE-safe animal-based protein and a vegetable-based protein. The stabilizer component provides for the stabilization of a vaccine throughout its storage and administration.

This application claims priority from copending provisional Application No. 60/705,508, filed Aug. 4, 2005, the entire disclosure of which is hereby incorporated by reference.

### FIELD OF THE INVENTION

The present invention relates to vaccine stabilizers, and more particularly to protein-based stabilizers for live and live-attenuated virus vaccines that are substantially safe from bovine spongiform encephalopathy (BSE) and transmissible spongiform encephalopathy (TSE). The present invention also relates to veterinary vaccine formulations containing stabilizers from vegetable sources, and animal sources which are substantially free of BSE and TSE.

### BACKGROUND OF THE INVENTION

Attenuated and live viral organisms used in vaccines are sensitive to changes in their environment and degrade when exposed to suboptimal conditions. Thus, vaccine stabilizers are used as agents added to liquid, frozen, or lyophilized vaccines to maintain vaccine potency and efficacy. Often, these stabilizers are incorporated with the vaccine during preparation, and stabilize the vaccine throughout the manufacturing, storage and administration thereof. Stabilizers impart greater shelf life to the vaccine until the vaccine is readied for administration. While vaccine stabilizers have been effective at extending a vaccine's potency throughout the manufacturing and storage process, new health concerns must be considered when choosing the type of stabilizer and its source.

Numerous methods are known for the production of live and attenuated viral vaccine preparations containing stabilizers. Traditional vaccine virus stabilizers are often comprised of animal proteins, along with sugars to stabilize the virus. Ruminant-derived proteins such as milk proteins, Pharmatone® and Peptone® stabilizers are commonly used in the stabilization process. Pharmatone® (American Labs, Inc., Omaha, Nebr.) is obtained by heat hydrolysis and peptic digestion of beef tissues. Bacto Peptone® (Difco Laboratories, Inc., Tucker, Ga.) is an enzymatic digest of animal proteins.

In addition, U.S. Pat. App. No. 2003/0215455 (Nov. 20, 2003) (B. Reynolds et al.) discloses a vaccine stabilizer that comprises a reducing agent, a buffer, a thermal stabilizer, a sugar and water. This reference also teaches to utilize a coloring agent to serve as a visual reference to an animal caretaker for positively identifying the presence of the vaccine in its own integral water supply for dissemination to an animal herd.

U.S. Pat. No. 5,733,555 (Mar. 31, 1998) (H. J. Chu) discloses a formulation comprising a modified virus combined with a stabilizer, carrier, or diluent. In addition, the formulation further comprises an adjuvant that is present in a final concentration of about 1-25% (v/v), and preferably 5% (v/v).

U.S. Pat. No. 6,258,362 (Jul. 10, 2001) (P. T. Loudon et al.) relates to a dried pharmaceutical composition dispersible in an aqueous liquid or an injection, containing a virus, a polysaccharide or a source of mixed amino acids, a buffer and a mono- or oligo-saccharide or derivatives thereof. However, the source of the amino acid is necessarily of vegetable or bacterial origin. Substantially TSE/BSE-safe protein or amino acid tissue sources such as cow's milk are not disclosed. Compositions include examples free from animal protein and its hydrosylate or other materials of animal origin.

As a direct result of TSE and related threats (i.e. BSE) in ruminant sources, alternative protein sources are presently being sought for use as vaccine stabilizers. It is generally believed that the highest amounts of infectivity of TSE/BSE are found in the brain and spinal cord of ruminant animals in the final stages of clinical disease. Scientists have found that different ruminant tissues contain different amounts of the BSE agent. Some tissues, such as skeletal muscle and milk, have never been shown to have much, if any, infectivity. However, other proteins and gelatins that may similarly be used as stabilizers in vaccines are sourced from other tissues, including cow bones. Because the slaughtering and butchering methods used to obtain tissues and prepare materials can affect the amount of infectivity that may be present, it appears that only proteins and sugars sourced from cow's milk could present little or no risk of TSE/BSE. In addition, non-ruminants such as plants and vegetables potentially present one of the only TSE/BSE risk-free sources of stabilizer proteins.

Thus, the drawbacks associated with the known animal vaccine stabilizers are twofold. The first is the use of animal-based proteins from sources that pose a potential TSE/BSE threat. The second is the use of BSE/TSE-safe vegetable-based proteins alone in vaccines that fail to include beneficial animal-based proteins of a substantially BSE/TSE-safe source.

In light of these observations, there is a need in the art to develop a vaccine stabilizer comprising proteins of substantially TSE/BSE-safe sources that include both vegetables and animals. There is also a need to develop a vaccine formulation for veterinary use which contains a stabilizer component derived from both vegetable sources, as well as from animal based sources that are substantially free to BSE or TSE.

### SUMMARY OF THE INVENTION

In accordance with the present invention, there is provided an animal vaccine formulation consisting essentially of a viral immunogen, and a stabilizer component containing an animal-based protein of a substantially BSE/TSE-safe source and a vegetable-based protein. Further, in accordance with the present invention, a substantially TSE/BSE-safe protein source would preferably be ruminant milk. Vegetable-based proteins may be sourced from a suitable vegetable or plant species as hereinafter described.

In another aspect, the present invention provides an animal vaccine that includes a stabilizer component as set forth above, and further includes a buffer and sugar. Although phosphates, carboxylates and bicarbonates are preferred, other suitable buffering agents may be used. The sugar source typically includes saccharides, but other moisture retainers such as carbohydrates and sugar alcohols may also be included.

In yet another aspect, the present invention provides a method for the preparation of an animal vaccine formulation. The preparation includes the formation of a stabilizer component that includes a vegetable-based protein in a diluent. An animal-based protein of a substantially TSE/BSE-safe source is added as part of the stabilizer component. Additionally, a buffer and a sugar may be added. The stabilizer component can also be sterilized, if desired. Subsequent to the sterilization of the stabilizer component, a vaccine formulation is achieved by the addition of a viral immunogen to the stabilizer component. Lyophilization of the vaccine formulation yields a stabilized vaccine suitable for extended storage periods.

As a further part of the invention, there is also provided a vaccine stabilizer formulation consisting essentially of a vegetable protein stabilizer and an animal protein stabilizer of a substantially TSE/BSE-safe source.

Further, objects and features of the invention will become apparent from the detailed description and the claims set forth herein below.

### DETAILED DESCRIPTION OF THE INVENTION

There is a need for an animal vaccine formulation containing a viral immunogen, together with a stabilizer containing an animal-based protein of a substantially BSE/TSE-safe source and a vegetable-based protein. The present invention is directed toward further solutions to address this need.

As used herein, “viral immunogen” will typically denote a live or attenuated virus, or portion thereof. An attenuated virus is a virus that has been altered, typically by passaging in tissue culture cells, to attenuate its ability to cause disease, but which maintains its ability to protect against disease or infection when administered to animals. Examples include, but are not limited to, live and live-attenuated immunogens from such animals as cows, horses, pigs, sheep, fowl, including chickens, turkeys and pigeons, dogs, cats, and other veterinary species, in particular mammals. Illustrative immunogens include, but are not limited to, Bordetella bronchiseptica, canine adenovirus type 2 (CAV-2), canine distemper, canine parainfluenza virus, canine parvovirus (CPv), feline calicivirus (FCV), feline rhinotracheitis, feline panleukopenia, porcine pseudorabies virus (PRV), equine arteritis (EAV), bovine rhinotracheitis virus, bovine parainfluenza virus (PI3), bovine respiratory syncytial virus (BRSV), bovine viral disease Type I and Type II (BVD Type I and Type II). Illustrative fowl immunogens include, but are not limited to, avian infectious bronchitis virus (IBV), Newcastle disease virus (NDV), Infectious Bursal Disease Virus (IBDV), Infectious Laryngotracheitis virus (ILT), Avian encephalomyelitis virus (AE), Avian poxvirus, or Avian Reovirus. The use of the formulations of the present invention for fowl immunogens is particularly advantageous in that such immunogens are typically supplied to the end user as lyophilized product, so that stabilizers are necessary to ensure full potency upon reconstitution prior to administration of the vaccine to the target animal.

The live or live attenuated immunogen pool may be neat or in solution, depending on the concentration of the immunogen stock. If the titer is high, it may need to be diluted to achieve the target concentration.

The viral immunogen is included as part of the vaccine formulation as set forth herein. Typically, the viral immunogen may be added as part of a stock solution for the immunogen of interest. The loading of the viral immunogen may vary, but will often be within the range of about 0.001 to 50% (w/v), preferably within the range of about 0.01 to 10%. The skilled artisan can optimize the amount of immunogen based on the particular attributes of the final vaccine formulation desired. For example, a more potent or stronger vaccine immunogen may require less concentration in the final vaccine formulation, for example. The quantity of vaccine immunogen set forth above in the vaccine formulation is not inclusive of any diluent (such as water), or other suspending means or vehicle (such as oil).

A further component of the vaccine formulation of the invention is a stabilizer component. “Stabilizer component” denotes a combination of an animal-based protein of a substantially BSE/TSE-safe source and a vegetable-based protein, which stabilizes the vaccine formulation throughout its storage and administration. The stabilizer component may comprise about 0.01-30% (w/v) of the animal vaccine formulation, more preferably about 0.05-15% (w/v), and even more preferably about 1-10% (w/v).

As part of the stabilizer component, there is an animal based protein of a substantially BSE/TSE-safe source. An “animal-based protein of a substantially BSE/TSE-safe source” refers to proteins that are sourced from ruminant milk, and other sources, for example the muscle meat, of an animal, particularly a mammal, that are likely to be free of contamination from BSE and TSE. Suitable animal-based proteins include, but are not limited to, digested protein extracts such as N-Z-Amine®, N-Z-Amine AS® and N-Z-Amine YT® (Sheffield Products Co., Norwich, N.Y.), which are casein enzymatic hydrosylates of bovine milk. N-Z-Amine YT® is the preferred animal-based protein for use in the present invention.

The animal-based protein may comprise about 0.001-15% (w/v) of the stabilizer component, more preferably about 0.01-10% (w/v) thereof. The animal-based protein will further comprise about 0.0001-3% (w/v), more preferably about 0.001-2% (w/v) of the vaccine formulation.

A further part of the stabilizer component is the vegetable-based protein. Vegetable-based proteins may include, without limitation, soy protein, wheat protein, corn gluten, rice protein and hemp protein, among others. Preferred vegetable based proteins in the present invention are soy proteins and corn gluten. Corn gluten is a mixture of various corn-derived proteins. The soy proteins can include 100% soy protein (available as VegeFuel® by Twinlab), textured soy protein, and soybean enzymatic digest. Textured soy protein is a soy protein that is made from defatted soy flour that is compressed and processed into granules or chunks. Soybean enzymatic digest describes soybean peptones that result from the partial hydrolysis of soybean proteins.

The vegetable-based protein may comprise about 0.001-30% (w/v) of the stabilizer component, more preferably about 0.01-10% (w/v) thereof. The vegetable-based protein will further comprise about 0.0001-6% (w/v), more preferably about 0.001-4% (w/v) of the final vaccine formulation. The ratio of vegetable-based protein to animal-based protein may preferably range from about 1:1 to about 3:1, and more preferably will be in the range of about 2:1.

A diluent, preferably water, will typically comprise the reminder of the stabilizer component, up to 100%.

The vaccine formulation of the invention may additionally include a biologically acceptable sugar for moisture retention during the lyophilization process, hereinafter described. The sugar is selected from the group including, but not limited to, the mono-, di-, tri- and oligosaccharides, such as glucose, dextrose, lactose, sucrose, mannose and fructose, and the like. Sucrose is the preferred sugar in the formulation of the present invention. The sugar may comprise about 0.001-6% (w/v) of the final vaccine formulation, more preferably about 0.001-4% (w/v). If desired, the sugar component may be included as part of the stabilizer component during preparation thereof. The skilled artisan may recognize that an approximate 1:1 ratio of sugar component to vegetable-based protein in the final formulation may be desirable.

The vaccine formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. Monosodium glutamate is the most preferred buffer as part of the present invention. The buffer may comprise about 0.0001-5% (w/v) of the vaccine formulation, more preferably about 0.001-1% (w/v). The buffer(s) may be added as part of the stabilizer component during the preparation thereof, if desired.

Other excipients, if desired, may be included as part of the final vaccine formulation.

The remainder of the vaccine formulation is an acceptable diluent, to 100%, including water. The vaccine formulation may also be formulated as part of a water-in-oil, or oil-in-water emulsion. The skilled artisan will further recognize that the heretofore described components comprising the vaccine formulation are an integral part thereof, and are not readily parseable therefrom.

Also provided as part of the invention is a method of preparation of the vaccine formulation herein described. Preparation of the vaccine formulation preferably takes place in two phases. The first phase typically involves the preparation of the stabilizer component. A vegetable-based protein stock solution is prepared by dissolving the vegetable-based protein in a diluent. The preferred diluent is water, preferably distilled and/or purified so as to remove trace impurities (such as that sold as purified Super Q®)). In a separate vessel an animal-based protein of a substantially BSE/TSE-safe source is dissolved in a diluent, additionally with the sugar component and buffer additives. N-Z-Amine YT® a high quality source of peptides produced by enzymatic hydrolysis of casein, is a preferred animal-based protein, while water is the preferred diluent. Preferably, an equal volume of the vegetable-based protein stock solution is added to the animal-based protein solution. It is desirable that after HCl/KOH adjustment to achieve a pH of approximately 7.2±0.1, the stabilizer component is sterilized via autoclave. The stabilizer solution may be refrigerated for extended period prior to introduction of the immunogen.

The second phase of preparation of the vaccine formulation includes introduction of the live or live attenuated immunogen with the stabilizer component, thereby yielding the vaccine formulation. Preferably, the immunogen is diluted with a buffer solution prior to its introduction to the stabilizer component.

Once this vaccine formulation solution has been achieved, the formulation is separated into vials or other suitable containers.

The vaccine formulation herein described may then be packaged in individual or multi-dose ampoules, or be subsequently lyophilized (freeze-dried) before packaging in individual or multi-dose ampoules. The vaccine formulation herein contemplated also includes the lyophilized version. The lyophilized vaccine formulation may be stored for extended periods of time without loss of viability at ambient temperatures.

The lyophilized vaccine may be reconstituted by the end user, and administered to an animal, typically in one or two doses, in the range of about 1-5 mL of vaccine formulation/dose. Smaller animals such as fowl may receive a preferred dosage of about 0.01-1 mL, more preferably about 0.03-0.5 mL.

The following example illustrates preferred aspects of the invention, but should not be construed as limiting the scope thereof:

### EXAMPLE 1

Step 1: Vegetable-Based Protein Stock Solution Preparation

5.0 g of corn gluten was mixed with 50 mL of purified water. The mixture was then ground in a blender. The mixture was decanted and the supernatant collected. The solution was stored at 4° C.

Step 2: Preparation of the Stabilizer Componet

N-Z-Amine Type YT® (2.5 g), Sucrose (5.0 g) (Sigma, St. Louis, Mo.) and Monosodium Glutamate (0.5 g) (Sigma, St. Louis, Mo.) were added to 50 mL of water (purified Super Q®) and dissolved. Heat was added to increase the dissolution rate. This solution was added to the 50.0 mL, 10% Corn Gluten solution of Step 1. The pH was adjusted to 7.2±0.1 with KOH and/or HCl. The solution was then dispensed into suitable containers and autoclaved at >121° C. or >30 minutes. (This solution may be stored at 2-7° C. for approximately 1 month.)

Step 3: Preparation and Lyophilization of the Vaccine Formulation

Preparation of the Virus Pool: A frozen avian infectious bronchitis virus (IBV), Newcastle disease virus (NDV), Infectious Bursal Disease Virus (IBDV), Infectious Laryngotracheitis virus (ILT), Avian encephalomyelitis virus (AE), Avian poxvirus, or Avian Reovirus preparation was or is thawed at 37° C. 50 mL of the thawed virus was or is added to a sterile bottle, diluted with 400 ml of PBS (phosphate buffered saline), and the viral titer recorded (EID₅₀/mL).

Preparation of the Vaccine Formulation: 15 mL of the stabilizer component (see Step 2 above) was or is added to a sterile bottle and diluted with 85 mL of the diluted viral immunogen of Step 3a. This vaccine formulation was or mixed for 5-10 minutes, and the theoretical immunogen titer recorded (EID₅₀/mL).

Aliquoting Immunogen/Stabilizer Pools: The vaccine formulation of Step 3b was or is dispensed in 5 mL aliquots to lyophilization bottles with sterile cornwalls and sterile blunt needles. Sterile stoppers were or are added to each vial and the vials added to the lyophilizer. After lyophilization, the vials were backfilled with 10-15% nitrogen, stoppered, crimp sealed, and stored at 4° C.

### EXAMPLE 2

Step 1: Vegetable-Based Protein Stock Solution Preparation

5.0 g of soy protein (VegeFuel® by Twinlab) was mixed with 50 mL of purified water. The mixture was then ground in a blender. The mixture was decanted and the supernatant collected. The solution was stored at 4° C.

Step 2: Preparation of the Stabilizer Componet

N-Z-Amine Type YT® (2.5 g) (Sheffield Products Co., Norwich, N.Y.), Sucrose (5.0 g) (Sigma, St. Louis, Mo.) and Monosodium Glutamate (0.5 g) (Sigma St. Louis, Mo.) were added to 50 mL of water (purified Super Q®) and dissolved. Heat was added to increase the dissolution rate. This solution was added to the 50.0 mL, 10% soy protein (VegeFuel® by Twinlab) of Step 1. The pH was adjusted to 7.2±0.1 with KOH and/or HCl. The solution was then dispensed into suitable containers and autoclaved at >121° C. or >30 minutes. (This solution may be stored at 2-7° C. for approximately 1 month.)

Step 3: Preparation and Lyophilization of the Vaccine Formulation

Preparation of the Virus Pool: A frozen avian infectious bronchitis virus (IBV), Newcastle disease virus (NDV), Infectious Bursal Disease Virus (IBDV), Infectious Laryngotracheitis virus (ILT), Avian encephalomyelitis virus (AE), Avian poxvirus, or Avian Reovirus preparation was or is thawed at 37° C. 50 mL of the thawed virus was or is added to a sterile bottle, diluted with 400 ml of PBS (phosphate buffered saline), and the viral titer recorded (EID₅₀/mL).

Preparation of the Vaccine Formulation: 15 mL of the stabilizer component (see Example 2 above) was or is added to a sterile bottle and diluted with 85 mL of the diluted viral immunogen of Step 3a. This vaccine formulation was or is mixed for 5-10 minutes, and the theoretical immunogen titer recorded (EID₅₀/mL).

Aliquoting Immunogen/Stabilizer Pools: The vaccine formulation of Step 3b was or is dispensed in 5 mL aliquots to lyophilization bottles with sterile cornwalls and sterile blunt needles. Sterile stoppers were or are added to each vial and the vials added to the lyophilizer. After lyophilization, the vials were or are backfilled with 10-15% nitrogen, stoppered, crimp sealed and stored at 4° C.

### EXAMPLE 3

Step 1: Vegetable-Based Protein Stock Solution Preparation

5.0 g of textured soy protein was mixed with 50 mL of purified water. The mixture was then ground in a blender. The mixture was decanted and the supernatant collected. The solution was stored at 4° C.

Step 2: Preparation of the Stabilizer Componet

N-Z-Amine Type YT® (2.5 g) (Sheffield Products Co., Norwich, N.Y.), Sucrose (5.0 g) (Sigma, St. Louis, Mo.) and Monosodium Glutamate (0.5 g) (Sigma St. Louis, Mo.) were added to 50 mL of water (purified Super Q®)) and dissolved. Heat was added to increase the dissolution rate. This solution was added to the 50.0 mL, 10% textured soy protein of Step 1. The pH was adjusted to 7.2±0.1 with KOH and/or HCl. The solution was then dispensed into suitable containers and autoclaved at >121° C. or >30 minutes. (This solution may be stored at 2-7° C. for approximately 1 month.)

Step 3: Preparation and Lyophilization of the Vaccine Formulation

Preparation of the Virus Pool: A frozen avian infectious bronchitis virus (IBV), Newcastle disease virus (NDV), Infectious Bursal Disease Virus (IBDV), Infectious Laryngotracheitis virus (ILT), Avian encephalomyelitis virus (AE), Avian poxvirus, or Avian Reovirus preparation was or is thawed at 37° C. 50 mL of the thawed virus was or is added to a sterile bottle, diluted with 400 ml of PBS (phosphate buffered saline), and the viral titer recorded (EID₅₀/mL).

Preparation of the Vaccine Formulation: 15 mL of the stabilizer component prepared as detailed in Step 2 above, was or is added to a sterile bottle and diluted with 85 mL of the diluted viral immunogen of Step 3a. This vaccine formulation was or is mixed for 5-10 minutes, and the theoretical immunogen titer recorded (EID₅₀/mL).

Aliquoting Immunogen/Stabilizer Pools: The vaccine formulation of Step 3b was or is dispensed in 5 mL aliquots to lyophilization bottles with sterile cornwalls and sterile blunt needles. Sterile stoppers were or are added to each vial and the vials added to the lyophilizer. After lyophilization, the vials were or are backfilled with 10-15% nitrogen, stoppered, crimp sealed and stored at 4° C.

### EXAMPLE 4

Step 1: Vegetable-Based Protein Stock Solution Preparation

5.0 g of soybean enzymatic digest was mixed with 50 mL of purified water. The mixture was then ground in a blender, decanted and the supernatant collected. The solution was stored at 4° C.

Step 2: Preparation of the Stabilizer Componet

N-Z-Amine Type YT® (2.5 g) (Sheffield Products Co., Norwich, N.Y.), Sucrose (5.0 g) (Sigma, St. Louis, Mo.) and Monosodium Glutamate (0.5 g) (Sigma St. Louis, Mo.) were or are added to 50 mL of water (purified Super Q®) and dissolved. Heat was or is added to increase the dissolution rate. This solution was or is added to the 50.0 mL, 10% soybean enzymatic digest solution of Step 1. The pH was adjusted to 7.2±0.1 with KOH and/or HCl. The solution was or is then dispensed into suitable containers and autoclaved at >121° C. or >30 minutes. (This solution may be stored at 2-7° C. for approximately 1 month.)

Step 3: Preparation and Lyophilization of the Vaccine Formulation

Preparation of the Virus Pool: A frozen avian infectious bronchitis virus (IBV), Newcastle disease virus (NDV), Infectious Bursal Disease Virus (IBDV), Infectious Laryngotracheitis virus (ILT), Avian encephalomyelitis virus (AE), Avian poxvirus, or Avian Reovirus preparation was or is thawed at 37° C. 50 mL of the thawed virus was or is added to a sterile bottle, diluted with 400 ml of PBS (phosphate buffered saline), and the viral titer recorded (EID₅₀/mL).

Preparation of the Vaccine Formulation: 15 mL of the stabilizer component prepared as detailed in Step 2 above, was added to a sterile bottle and diluted with 85 mL of the diluted viral immunogen of Step 3a. This vaccine formulation was or is mixed for 5-10 minutes, and the theoretical immunogen titer recorded (EID₅₀/mL).

Aliquoting Immunogen/Stabilizer Pools: The vaccine formulation of Step 3b was dispensed in 5 mL aliquots to lyophilization bottles with sterile cornwalls and sterile blunt needles. Sterile stoppers were added to each vial and the vials were added to the lyophilizer. After lyophilization, the vials were backfilled with 10-15% nitrogen and then stoppered. The vials were then crimp sealed and stored at 4° C.

### EXAMPLE 5

### Comparison Testing with Animal-Based Stabilizer

Vaccine strain IBV harvest fluids were formulated at equivalent titer into stabilizers as described above in Examples 1-4, and compared to virus stabilized in animal protein in the manner of Poulvac® IB (Massachusetts, M-41—VS Code 1231.11) commercially available from by Fort Dodge Animal Health Division of Wyeth. Samples were obtained after the virus was mixed with stabilizer, and again after lyophilization cycling, were titrated in triplicate, and the pre- and post-lyophilization geometric mean titers were compared to determine loss during freeze drying.

Results are as follows:

| | Geometric | Geometric | |
|---|---|---|---|
| | Mean Titer | Mean Titer | Titer Loss |
| Sample | Pre-Lyo (log/mL) | Post-Lyo (log/mL) | (log/mL) |
| IBV + Example 1 | 6.20 | 5.54 | 0.66 |
| Stabilizer | | | |
| IBV + Example 2 | 5.40 | 4.66 | 0.74 |
| Stabilizer | | | |
| IBV + Example 3 | 5.80 | 4.61 | 1.19 |
| Stabilizer | | | |
| IBV + Example 4 | 5.80 | 4.78 | 1.02 |
| Stabilizer | | | |
| IBV + Animal | 5.70 | 3.65 | 2.05 |
| Protein Stabilizer | | | |

The use of the vegetable origin stabilizers of Examples 1-4 in the Table above resulted in lower titer loss during the lyophilization cycle than did use of the animal protein stabilizer (line 5 in the table above).

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. It is intended that the present invention be limited only to the extent required by the appended claims and the applicable rules of law.

What is claimed is:

1. An animal vaccine formulation consisting essentially of:
a live viral immunogen; and
a stabilizer component containing an animal-based protein of a substantially BSE/TSE-safe source and a vegetable-based protein.

2. The animal vaccine formulation of claim 1, wherein said live viral immunogen is attenuated.

3. The stabilizer component of claim 1, wherein said vegetable-based protein is selected from a group comprising corn gluten, soy protein, rice protein, wheat protein, and hemp protein.

4. The stabilizer component of claim 1, wherein said animal-based protein of a substantially BSE/TSE-safe source is N-Z-Amine®.

5. The animal vaccine formulation of claim 1, further containing:
a diluent.

6. The animal vaccine formulation of claim 5, wherein said diluent is purified water.

7. The animal vaccine formulation of claim 1, further containing:
a sugar.

8. The animal vaccine formulation of claim 7, wherein said sugar is selected from the group consisting of mono-, di-, tri- and oligosaccharides, such as glucose, dextrose, lactose, sucrose, mannose and fructose.

9. The animal vaccine of claim 8, wherein said sugar is sucrose.

10. The animal vaccine formulation of claim 1, further containing:
a buffer.

11. The animal vaccine formulation of claim 10, wherein said buffer is selected from a group including sodium phosphate, potassium phosphate, sodium citrate, calcium lactate, sodium succinate, sodium glutamate, sodium bicarbonate, and potassium bicarbonate.

12. The animal vaccine formulation of claim 11, wherein said buffer is monosodium glutamate.

13. An animal vaccine formulation consisting essentially of:
a live viral immunogen;
a stabilizer component containing an animal-based protein of a substantially BSE/TSE-safe source, and a vegetable-based protein;
a diluent;
a sugar; and
a buffer.

14. The animal vaccine formulation of claim 13, wherein said live viral immunogen is attenuated.

15. The animal vaccine formulation of claim 13 wherein said diluent is purified water.

16. The animal vaccine formulation of claim 13, wherein said sugar is selected from a group including mono-, di-, tri- and oligosaccharides, such as glucose, dextrose, lactose, sucrose, mannose and fructose.

17. The animal vaccine formulation of claim 16, wherein said sugar is sucrose.

18. The animal vaccine formulation of claim 13, wherein said buffer is selected from the group consisting of sodium phosphate, potassium phosphate, sodium citrate, calcium lactate, sodium succinate, sodium glutamate, sodium bicarbonate, and potassium bicarbonate.

19. The animal vaccine formulation of claim 18, wherein said buffer is monosodium glutamate.

20. The stabilizer component of claim 13, wherein said animal protein of a substantially BSE/TSE-safe source is N-Z-Amine Type YT®.

21. The stabilizer component of claim 13, wherein said vegetable-based protein is selected from a group comprising corn gluten, soy protein, rice protein, wheat protein, and hemp protein.

22. An animal vaccine formulation consisting essentially of:
a live viral immunogen;
a stabilizer component containing an animal protein of a substantially BSE/TSE-safe source and a vegetable-based protein, wherein said animal protein of a substantially BSE/TSE-safe source is N-Z-Amine Type YT® and said vegetable-based protein is selected from a group comprising corn gluten, soy protein, rice protein, wheat protein, and hemp protein;
a diluent, wherein said diluent is purified water;
a sugar, wherein said sugar is selected from a group consisting of mono-, di-, tri- and oligosaccharides, such as glucose, dextrose, lactose, sucrose, mannose and fructose; and
a buffer, wherein said buffer is selected from the group consisting of sodium phosphate, potassium phosphate, sodium citrate, calcium lactate, sodium succinate, sodium glutamate, sodium bicarbonate, and potassium bicarbonate.

23. The animal vaccine formulation of claim 22, wherein said sugar is sucrose.

24. The animal vaccine formulation of claim 22, wherein said buffer is monosodium glutamate.

25. The animal vaccine formulation of claim 22, wherein said live viral immunogen is attenuated.

26. A method of preparing an animal vaccine formulation, comprising:
forming a stabilizer component consisting essentially of an animal-based protein of a substantially BSE/TSE-safe source and vegetable-based protein; and
adding said stabilizer component to an immunogen component to furnish a vaccine formulation.

27. The method set forth in claim 26, wherein said vegetable-based protein comprises about 0.0001-6% (w/v), more preferably about 0.001-4% (w/v) of said vaccine formulation.

28. The method set forth in claim 26, wherein said animal-based protein comprises about 0.0001-3% (w/v), more preferably about 0.001-2% (w/v) of said vaccine formulation.

29. The method set forth in claim 26, wherein said vaccine formulation is lyophilized after said adding of said immunogen component.

30. The method set forth in claim 26, wherein the preparation of said stabilizer component of said vaccine formulation comprises:
dissolving said vegetable-based protein in purified water to form a first solution;
adding said animal-based protein of a substantially BSE/TSE-safe source, said buffer, and said sugar to said first solution;
adjusting the pH of said stabilizer solution;
filtering said stabilizer component; and
sterilizing said stabilizer component.

31. The method set forth in claim 30, wherein said animal-based protein of a substantially BSE/TSE-safe source is N-Z-Amine Type YT®, said buffer is monosodium glutamate, and said sugar is sucrose.

32. The method set forth in claim 30, wherein said vegetable-based protein is selected from the group consisting of corn gluten, soy protein, rice protein, wheat protein, and hemp protein.

33. The method set forth in claim 30, wherein said vegetable-based protein of said stabilizer component comprises about 0.001-30% (w/v) of the stabilizer component, more preferably about 0.01-10% (w/v).

34. The method set forth in claim 30, wherein said animal-based protein of said stabilizer component comprises about 0.001-15% (w/v) of the stabilizer component, more preferably about 0.01-10% (w/v) thereof.

35. The method set forth in claim 30, wherein said sugar comprises about 0.001-6% (w/v) of the final vaccine formulation, more preferably about 0.001-4% (w/v).

36. The method set forth in claim 30, wherein said buffer comprises 0.0001-5% (w/v) of the vaccine formulation, more preferably about 0.001-1% (w/v).

37. The method set forth in claim 30, wherein said dissolving further comprises:
heating said first solution.

38. The method set forth in claim 30, wherein said sterilizing comprises autoclaving said solution.

39. A vaccine stabilizer consisting essentially of:
animal-based protein of a substantially BSE/TSE-safe source; and
a vegetable-based protein.

40. The vaccine stabilizer component of claim 39, wherein the ratio of said vegetable-based protein to said animal-based protein is within the range of from about 1:1 to about 3:1.
